SEQUENCE LISTING (21 sequences)

SEQ ID NO: 1 - DNA, 348 bp, Mus musculus
  source 1..348: /mol_type=“DNA” /organism=“Mus musculus”
gcggtccagctgcagcagtctggacctgagctggaaaagcctggcgcttcagtgaagatttcctgcaaggcttctggttactcattcactggctacaatatgaactgggtgaagcagaataatggaaagagccttgagtggattggaaatattgatccttattatggtggtactacctacaaccggaagttcaagggcaaggccacattgactgtagacaaatcctccagcacagcctacatgcagctcaagagtctgacatctgaggactctgcagtctattactgtgcaagatcggtcggccctatggactactggggtcaaggaacctcagtcaccgtctcttct

SEQ ID NO: 2 - protein, 116 aa, Mus musculus
  SOURCE 1..116: /mol_type=“protein” /organism=“Mus musculus”
AVQLQQSGPELEKPGASVKISCKASGYSFTGYNMNWVKQNNGKSLEWIGNIDPYYGGTTYNRKFKGKATLTVDKSSSTAYMQLKSLTSEDSAVYYCARSVGPMDYWGQGTSVTVSS

SEQ ID NO: 3 - DNA, 324 bp, Mus musculus
  source 1..324: /mol_type=“DNA” /organism=“Mus musculus”
gacatccagatgactcagtctccagcctccctatctgcatctgtgggagagactgtcaccatcacatgtcgaacaagtgaaaatgtttacagttatttggcttggtatcagcagaaacagggaaaatctcctcagctcctggtctcttttgcaaaaaccttagcagaaggtgtgccatcaaggttcagtggcagtggatcaggcacacagttttctctgaagatcagcagcctgcagcctgaagattctggaagttatttctgtcaacatcattccgataatccgtggacgttcggtggaggcaccgaactggagatcaaacga

SEQ ID NO: 4 - protein, 108 aa, Mus musculus
  SOURCE 1..108: /mol_type=“protein” /organism=“Mus musculus”
DIQMTQSPASLSASVGETVTITCRTSENVYSYLAWYQQKQGKSPQLLVSFAKTLAEGVPSRFSGSGSGTQFSLKISSLQPEDSGSYFCQHHSDNPWTFGGGTELEIKR

SEQ ID NO: 5 - protein, 446 aa, Artificial Sequence
  SOURCE 1..446: /mol_type=“protein” /note=“Synthetic construct heavy chain A2” /organism=“Artificial Sequence”
AVQLQQSGPELEKPGASVKISCKASGYSFTGYNMNWVKQNNGKSLEWIGNIDPYYGGTTYNRKFKGKATLTVDKSSSTAYMQLKSLTSEDSAVYYCARSVGPMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, Artificial Sequence
  SOURCE 1..214: /mol_type=“protein” /note=“Synthetic construct light chain A2” /organism=“Artificial Sequence”
DIQMTQSPASLSASVGETVTITCRTSENVYSYLAWYQQKQGKSPQLLVSFAKTLAEGVPSRFSGSGSGTQFSLKISSLQPEDSGSYFCQHHSDNPWTFGGGTELEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 330 aa, Artificial Sequence
  SOURCE 1..330: /mol_type=“protein” /note=“Synthetic construct constant heavy chain” /organism=“Artificial Sequence”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 106 aa, Artificial Sequence
  SOURCE 1..106: /mol_type=“protein” /note=“Synthetic construct constant light chain” /organism=“Artificial Sequence”
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 446 aa, Artificial Sequence
  SOURCE 1..446: /mol_type=“protein” /note=“Synthetic construct heavy chain A4” /organism=“Artificial Sequence”
AVQLQQSGPELEKPGASVKISCKASGYSFTGYNMNWVKQNNGKSLEWIGNIDPYYGGTTYNRKFKGKATLTVDKSSSTAYMQLKSLTSEDSAVYYCARSVGPMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial Sequence; identical to SEQ ID NO: 6
  SOURCE 1..214: /mol_type=“protein” /note=“Synthetic construct light chain A4” /organism=“Artificial Sequence”

SEQ ID NO: 11 - protein, 446 aa, Artificial Sequence
  SOURCE 1..446: /mol_type=“protein” /note=“Synthetic construct heavy chain B2” /organism=“Artificial Sequence”
AVQLVQSGAEVKKPGSSVKVSCKASGYSFTGYNMNWVRQAPGQGLEWMGNIDPYYGGTTYNRKFKGRVTLTVDKSSSTAYMELSSLRSEDTAVYYCARSVGPMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 214 aa, Artificial Sequence
  SOURCE 1..214: /mol_type=“protein” /note=“Synthetic construct light chain B2” /organism=“Artificial Sequence”
DIQMTQSPSSLSASVGDRVTITCRTSENVYSYLAWYQQKPGKAPKLLVSFAKTLAEGVPSRFSGSGSGTDFTLTISSLQPEDFATYFCQHHSDNPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 446 aa, Artificial Sequence
  SOURCE 1..446: /mol_type=“protein” /note=“Synthetic construct heavy chain B4” /organism=“Artificial Sequence”
AVQLVQSGAEVKKPGSSVKVSCKASGYSFTGYNMNWVRQAPGQGLEWMGNIDPYYGGTTYNRKFKGRVTLTVDKSSSTAYMELSSLRSEDTAVYYCARSVGPMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 214 aa, Artificial Sequence; identical to SEQ ID NO: 12
  SOURCE 1..214: /mol_type=“protein” /note=“Synthetic construct light chain B4” /organism=“Artificial Sequence”

SEQ ID NO: 15 - protein, 5 aa, Artificial Sequence
  SOURCE 1..5: /mol_type=“protein” /note=“CDR1 heavy chain (H1)” /organism=“Artificial Sequence”
GYNMN

SEQ ID NO: 16 - protein, 17 aa, Artificial Sequence
  SOURCE 1..17: /mol_type=“protein” /note=“CDR2 heavy chain (H2)” /organism=“Artificial Sequence”
NIDPYYGGTTYNRKFKG

SEQ ID NO: 17 - protein, 7 aa, Artificial Sequence
  SOURCE 1..7: /mol_type=“protein” /note=“CDR3 heavy chain (H3)” /organism=“Artificial Sequence”
SVGPMDY

SEQ ID NO: 18 - protein, 11 aa, Artificial Sequence
  SOURCE 1..11: /mol_type=“protein” /note=“CDR1 light chain (L1)” /organism=“Artificial Sequence”
RTSENVYSYLA

SEQ ID NO: 19 - protein, 7 aa, Artificial Sequence
  SOURCE 1..7: /mol_type=“protein” /note=“CDR2 light chain (L2)” /organism=“Artificial Sequence”
FAKTLAE

SEQ ID NO: 20 - protein, 9 aa, Artificial Sequence
  SOURCE 1..9: /mol_type=“protein” /note=“CDR3 light chain (L3)” /organism=“Artificial Sequence”
QHHSDNPWT

SEQ ID NO: 21 - protein, 13 aa, Artificial Sequence
  SOURCE 1..13: /mol_type=“protein” /note=“alternative CDR2 heavy chain (H2b)” /organism=“Artificial Sequence”
NIDPYYGGTTYNR

(19) United States
(12) Patent Application Publication
(10) Pub. No.: US 2016/0106837 A1
(43) Pub. Date: Apr. 21, 2016

(54) COMBINATION OF CD37 ANTIBODIES WITH CHLORAMBUCIL

(71) Applicant: BOEHRINGER INGELHEIM INTERNATIONAL GMBH, Ingelheim Am Rhein (DE)

(72) Inventors: Karl-Heinz HEIDER, Stockerau (AT); Petra BLUM, Biberach an der Riss (DE)

(21) Appl. No.: 14/897,749

(22) PCT Filed: Jun. 14, 2013
(86) PCT No.: PCT/EP2013/062365

§ 371 Date: Dec. 11, 2015

(51) Int. Cl. A61K 39/395; A61K 31/196

(52) U.S. Cl. CPC A61K 39/39558; A61K 31/196; A61K 2039/545

(57) ABSTRACT

The present invention relates to immunotherapies that are based on depletion of CD37-positive cells such as B-cells. The present invention provides methods for reduction of CD37-positive cells such as B-cells in an individual/patient using a combination of CD37 antibody/antibodies and chlorambucil. The combination of CD37 antibodies and chlorambucilis shown to have a synergistic effect. The application further provides materials and methods for treatment of diseases involving aberrant B-cell activity.

### BACKGROUND OF THE INVENTION

1. Technical Field

The present invention relates to immunotherapies that are based on depletion of CD37-positive cells such as B-cell cells. In particular, the present invention relates to a combination of CD37 antibodies, especially A2 and B2, with chemotherapy, especially chlorambucil for use in such therapies, e.g. in the treatment of B-cell malignancies, other CD37-positive malignancies, and autoimmune conditions.

2. Background

Immunotherapy using monoclonal antibodies (mAbs) has emerged as a safe and selective method for the treatment of cancer and other diseases. In particular, the role of monoclonal antibodies in therapies that are based on B-cell depletion, e.g. in the treatment of B-cell malignancies, has expanded since the introduction of rituximab (Rituxan®), an antibody that is directed against the CD20 antigen on the B-cell surface. Numerous studies have confirmed the efficacy of rituximab as a single agent and in combination therapy in low-grade NHL. Frontline therapy with rituximab added to the combination of cyclophosphamide, doxorubicin, vincristine, and prednisolone (CHOP) significantly improves the outcome for patients with advanced-stage follicular lymphoma compared with therapy with CHOP alone (Hiddemann W, et al. Blood 2005; 106: 3725-3732 (2005))The addition of rituximab to a combination of fludarabine, cyclophosphamide, mitoxantrone (FCM) significantly increases the response rate and prolongs survival as compared with FCM alone in patients with relapsed and refractory follicular and mantle cell lymphomas (Forstpointner R, et al., Blood, 2004; 104: 3064-3071).

However, only a subset of patients responds to therapy and the majority of those eventually relapse following rituximab treatment. Therefore, there is a need to find immunotherapies with higher efficacy than antibodies that are directed against the CD20 antigen (rituximab).

### SUMMARY OF THE INVENTION

The invention describes CD37 antibodies, preferably A2 and B2, used in combination with chlorambucil. This combination surprisingly results in a synergistic anti-tumor effect. The two therapeutic agents, CD37 antibody and chlorambucil, may be administered simultaneously, optionally as a component of the same pharmaceutical preparation, or chlorambucil may be administered before or after administration of the CD37 antibody.

s In accordance with the invention, there are provided novel combinations of anti-CD37 antibodies as described in the present invention with chlorambucil. Accordingly, the combination of anti-CD37 antibodies of the present invention and chlorambucil are used to treat patients suffering from B-cell malignancies.

A high degree of tumor cell killing in patients with B-cell malignancies, e.g. CLL and B-NHL, is considered advantageous for the treatment of those patients and is considered to translate into increased clinical benefit for patients treated with such an agent. In a mice model of human follicular lymphoma (DOHH2), CD37 antibodies such as A2 in combination with chlorambucil induced tumor regression in all animals, and importantly is statistical analysis indicated a synergistic enhancement of efficacy when compared to single agent therapy.

The benefit of a combination treatment with CD37 antibodies, especially mAbs A2 or B2, and a chemotherapeutic agent such as chlorambucil can be further demonstrated in clinical trials, which compare the efficacy of chlorambucil monotherapy against the efficacy of a combination of chlorambucil and CD37 antibodies, especially mAb A2 or B2. The trial is performed in a randomized fashion, e.g. the patients are assigned to the two different treatment arms of the study in a randomized fashion. The response to treatment is defined by standardized response criteria for the respective indication. The efficacy of the treatment is assessed by surrogate parameters like progression free survival (PFS) or response rate. A clinically relevant therapeutic effect is for example the prolongation of PFS by 50% with chlorambucil and A2 or B2 compared to chlorambucil alone (e.g. 27 months PFS compared to 18 months) or an increase in complete response rate by 50% with chlorambucil and A2 or B2 compared to chlorambucil alone (e.g. 45% compared to 30%) for patients with CD37 positive malignancies like mature B-cell malignancies, e.g. relapsed chronic lymphocytic leukemia.

Furthermore, the benefit of a combination treatment with CD37 antibodies, especially mAbs A2 or B2, a chemotherapeutic agent such as chlorambucil, and a CD20 antibody such as Rituximab can be further demonstrated in clinical trials, which compare the efficacy of chlorambucil administered in combination with anti-CD20 antibody (e.g. Rituximab), which is referred to as R-chlorambucil, against the efficacy of a combination of chlorambucil administered in combination with anti-CD20 antibody and additionally CD37 antibody, especially mAb A2 or B2. Such a trial is performed in a randomized fashion, e.g. the patients are assigned to the two different treatment arms of the study by randomization. The response to treatment is defined by standardized response criteria for the respective indication. The efficacy of the treatment is assessed by surrogate parameters like progression free survival (PFS) or response rate. A clinically relevant therapeutic effect is for example the prolongation of PFS by 50% with R-chlorambucil and A2 or B2 compared to R-chlorambucil alone (e.g. 27 months PFS compared to 18 months) or an increase in complete response rate by 50% with R-chlorambucil and A2 or B2 compared is to R-chlorambucil alone (e.g. 45% compared to 30%) for patients with CD37 positive malignancies like mature B-cell malignancies, e.g. relapsed chronic lymphocytic leukemia.

To be used in therapy, the CD37 antibody is included into pharmaceutical compositions appropriate to facilitate administration to animals or humans. Typical formulations of the CD37 antibody molecule can be prepared by mixing the CD37 antibody molecule with physiologically acceptable carriers, excipients or stabilizers, in the form of lyophilized or otherwise dried formulations or aqueous solutions or aqueous or non-aqueous suspensions.

Pharmaceutically acceptable carriers and adjuvants for use with CD37 antibodies according to the present invention include, for example, ion exchangers, alumina, aluminum stearate, lecithin, serum proteins, buffer substances, water, salts or electrolytes and cellulose-based substances.

Carriers, excipients, modifiers or stabilizers are nontoxic at the dosages and concentrations employed. They include buffer systems such as phosphate, citrate, acetate and other anorganic or organic acids and their salts; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone or polyethylene glycol (PEG); amino acids such as glycine, glutamine, asparagine, histidine, arginine, or lysine; monosaccharides, disaccharides, oligosaccharides or polysaccharides and other carbohydrates including glucose, mannose, sucrose, trehalose, dextrins or dextrans; chelating agents such as EDTA; sugar alcohols such as, mannitol or sorbitol; salt-forming counter-ions such as sodium; metal complexes (e.g. Zn-protein complexes); and/or ionic or non-ionic surfactants such as TWEEN™ (polysorbates), PLURONICS™ or fatty acid esters, fatty acid ethers or sugar esters. Also organic solvents can be contained in the antibody formulation such as ethanol or isopropanol. The excipients may also have a release-modifying or absorption-modifying function. This is not a complete list of possible pharmaceutically acceptable carriers and adjuvants, and one of ordinary skilled in the art would know other possibilities, which are replete in the art.

As further explained in Example 1 below, in one embodiment the CD37 antibody A2 is formulated in a vehicle containing 25 mM Na-citrate, 125 mM NaCl, 0.02% PS20 pH 6.2 and diluted with PBS.

The CD37 antibody molecules may also be dried (freeze-dried, spray-dried, spray-freeze dried, dried by near or supercritical gases, vacuum dried, air-dried), precipitated or crystallized or entrapped in microcapsules that are prepared, for example, by coacervation techniques or by interfacial polymerization using, for example, hydroxymethylcellulose or gelatin and poly-(methylmethacylate), respectively, in colloidal drug delivery systems (for example, liposomes, albumin microspheres, microemulsions, nano-particles and nanocapsules), in macroemulsions or precipitated or immobilized onto carriers or surfaces, for example by pcmc technology (protein coated microcrystals). Such techniques are known in the art.

Naturally, the pharmaceutical compositions/formulations to be used for in vivo administration must be sterile; sterilization may be accomplished be conventional techniques, e.g. by filtration through sterile filtration membranes.

It may be useful to increase the concentration of the CD37 antibody to come to a so-called high concentration liquid formulation (HCLF); various ways to generate such HCLFs have been described.

The CD37 antibody molecule may also be contained in a sustained-release preparation. Such preparations include solid, semi-solid or liquid matrices of hydrophobic or hydrophilic polymers, and may be in the form of shaped articles, e.g. films, sticks or microcapsules and may be applied via an application device. Examples of sustained-release matrices include polyesters, hydrogels (for example, poly(2-hydroxyethyl-methacrylate) or sucrose acetate butyrate), or poly(vinylalcohol)), polylactides (U.S. Pat. No. 3,773,919), copolymers is of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(-)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S-S bond formation through thio-disulfide interchange, stabilization may be achieved by modifying sulfhydryl residues, lyophilization from acidic solutions, controlling moisture content, using appropriate additives, and developing specific polymer matrix compositions.

The CD37 antibody molecule, especially A2 and B2, can be incorporated also in other application forms, such as dispersions, suspensions or liposomes, tablets, capsules, powders, sprays, transdermal or intradermal patches or creams with or without permeation enhancing devices, wafers, nasal, buccal or pulmonary formulations, or may be produced by implanted cells or—after gene therapy—by the individual's own cells.

A CD37 antibody molecule, especially A2 and B2, may also be derivatized with a chemical group such as polyethylene glycol (PEG), a methyl or ethyl group, or a carbohydrate group. These groups may be useful to improve the biological characteristics of the antibody, e.g. to increase serum half-life or to increase tissue binding.

The preferred mode of application is parenteral, by infusion or injection (intravenous, intramuscular, subcutaneous, intraperitoneal, intradermal), but other modes of application such as by inhalation, transdermal, intranasal, buccal, oral, may also be applicable.

For therapeutic use, the compounds may be administered in a therapeutically effective amount in any conventional dosage form in any conventional manner. Routes of administration include, but are not limited to, intravenously, intramuscularly, subcutaneously, intrasynovially, intrathecally by infusion, sublingually, transdermally, orally, topically or by inhalation, tablet, capsule, caplet, liquid, solution, suspension, emulsion, lozenges, syrup, reconstitutable powder, granule, suppository and transdermal patch. Methods for preparing such dosage forms are known (see, for example, H. C. Ansel and N. G. Popovish, Pharmaceutical Dosage Forms and Drug Delivery Systems, 5th ed., Lea and Febiger (1990)). A therapeutically effective amount can be determined by a skilled artisan based upon such factors as weight, metabolism, and severity of the affliction etc.

Preferably the active compound is dosed at about 0.01 μg to about 500 μg per kilogram of body weight at least once per treatment cycle, e.g. on a weekly basis (0.01μg to 500 mg per kilogram of body weight). More preferably the active compound is dosed at about 0.01 mg to 40 mg per kilogram of body weight at least once per treatment cycle.

For the prevention or treatment of disease, the appropriate dosage of antibody will depend on the type of disease to be treated, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

Depending on the type and severity of the disease, about 0.01 μg/kg to 40 mg/kg of CD37 antibody, especially of A2 and B2, is an initial candidate dosage for administration to the patient, whether, for example, by one or more separate administrations, or by infusion such as continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays, e.g. by determining the extent of B-cell depletion (e.g. using flow cytometry).

For A2, the estimated weekly dose for a 70 kg human is in the range of 1 mg to 2800 mg, is preferably 1 mg to 400 mg weekly or 2 mg to 800 mg every 2 weeks. The estimated human weekly dose for B2 for a 70 kg human is in the range of 1 mg to 2800 mg, preferably 1 mg to 1000 mg, e.g. 100 mg to 385 mg weekly or 200 mg to 770 mg every two weeks for a 70 kg person.

Treatment cycle: The treatment cycle is a time period of between 1 to 6 weeks, preferably 3 to 4 weeks, most preferably 4 weeks, wherein the patient receives at least one dose of the CD37 antibody and at least one dose of chlorambucil.

For CLL a preferred treatment cycle scheme lasts for a time period of 3 to 6 weeks, whereby chlorambucil is preferably administered daily at a dose of 0.1 to 0.2 mg/kg and whereby at least one CD37 antibody, preferably A2 or B2, is administered at a dose as described above either before, after or simultaneously with the chlorambucil administration. Simultaneously hereby means on the same day. Simultaneously furthermore may mean within six hours of each other or within one hour of each other or with the same injection. Furthermore, another preferred treatment cycle scheme for CLL comprises additional administration(s) of CD37 antibody in-between, for example in the middle of the treatment cycle at about 2 weeks.

Also for CLL, an alternative prefered treatment cycle scheme lasts for a time period of 4 weeks, whereby chlorambucil is preferably administered at a dose of 100 mg/m² body surface preferably on day 1 and 2 and whereby at least one CD37 antibody, preferably A2 or B2, is administered at a dose as described above either before, after or simultaneously with the chlorambucil administration. Simultaneously hereby means on the same day. Simultaneously furthermore may mean within six hours of each other or within one hour of each other or with the same injection. Furthermore, another prefered treatment cycle scheme for CLL comprises additional administration(s) of CD37 antibody inbetween, for example in the middle of the treatment cycle at about 2 weeks.

For NHL a preferred treatment cycle scheme lasts for a time period of 3 to 6 weeks, whereby chlorambucil is preferably administered daily at a dose of 0.1 to 0.2 mg/kg and whereby at least one CD37 antibody, preferably A2 or B2, is administered at a dose as is described above either before, after or simultaneously with the chlorambucil administration. Simultaneously hereby means on the same day. Simultaneously furthermore may mean within six hours of each other or within one hour of each other or with the same injection. Furthermore, another preferred treatment cycle scheme for NHL comprises additional administration(s) of CD37 antibody in-between, for example once a week, thus resulting in several, preferably 3 to 4 administrations of CD37 antibody per treatment cycle.

Also for NHL, an alternative prefered treatment cycle scheme lasts for a time period of 3 weeks, whereby chlorambucil is preferably administered at a dose of 120 mg/m² body surface preferably on day 1 and 2 and whereby at least one CD37 antibody, preferably A2 or B2, is administered at a dose as described above either before, after or simultaneously with the chlorambucil administration. Simultaneously hereby means on the same day. Simultaneously furthermore may mean within six hours of each other or within one hour of each other or with the same injection. Furthermore, another prefered treatment cycle scheme for NHL comprises additional administration(s) of CD37 antibody inbetween, for example once a week, thus resulting in several, preferably 3 to 4 administrations of CD37 antibody per treatment cycle.

Chlorambucil is preferably dosed daily on a treatment cycle, which is preferably 3-6 weeks (=21-36 days) long. The dose for chlorambucil ranges between 0.0.5-0.4 mg/kg. Preferably the dose ranges between 0.1-0.3 mg/kg. Alternate schedules for the treatment of CLL employing intermittent, biweekly, or once-monthly pulse doses of chlorambucil can be used. Intermittent schedules of chlorambucil begin with an initial single dose of 0.4 mg/kg. Doses are generally increased by 0.1 mg/kg until control of lymphocytosis or toxicity is observed. Subsequent doses are modified to produce mild hematologic toxicity.

Alternatively, chlorambucil is preferably dosed on two consecutive days (e.g. d1+d2) of a treatment cycle, which is preferably 3-4 weeks (=21 28 days) long. The dose for chlorambucil ranges between 50-150 mg/m2 body surface on 2 treatment days of a 3 to 4 week long treatment cycle. Preferably the dose ranges between 70-120 mg/m2 body surface or between 100-150 mg/m2 body surface on d1+2 of a treatment cycle. For the is treatment of a CLL patient chlorambucil is preferably administered at a dosage of 100 mg/m2 body surface on days 1 and 2 of the treatment cycle (e.g. 3-4 weeks, preferably 4 weeks). For the treatment of a NHL patient chlorambucil is preferably administered at a dosage of 120 mg/m2 body surface on days 1 and 2 of the treatment cycle (e.g. 3-4 weeks, preferably 3 weeks). Furthermore preferred is a dose in the range of 60-70 mg/m² body surface on d1+2 of a treatment cycle. But also a one-time administration of chlorambucil may be administered per treatment cycle with a somewhat higher dose (e.g. 140-400 mg/m2).

The chlorambucil dose is administered preferably on day 1 and on day 2 of a 3-4 week treatment cycle. Furthermore, preferred is the administration of chlorambucil on the 2 days following a CD37 antibody administration (e.g. day1=CD37 administration in any of the dosages as described above, days 2-3=chlorambucil administration in any of the dosages as described above) of a 3-4 week treatment cycle.

The chlorambucil dose may be administered by any way, e.g. infusion, parenteral or oral administration.

In another treatment cycle scheme chlorambucil is given together with Rituximab or another antibody targeting CD20. This treatment option is referred to as R-chlorambucil. In a preferred treatment cycle scheme for R-chlorambucil the Rituximab (or alternatively any other antibody targeting CD20) is embedded into the chlorambucil treatment cycle and dosing scheme (schemes/treatment cycles as described in the paragraphs above), preferably by administering the antibody targeting CD20 (e.g. Rituximab) together with chlorambucil on the 1^{st} treatment day. Alternatively, in another preferred treatment scheme the antibody targeting CD20 (e.g. Rituximab) is administered before the first chlorambucil administration on day1 of the treatment cycle (chlorambucil on days 2 and 3). A preferred dose for Rituximab is 100-500 mg/m² body surface, preferably 375-500 mg/m², most preferably 375 mg/m².

For CD37 combination therapy during the chlorambucil or R-chlorambucil treatment cycle at least one CD37 antibody, preferably A2 or B2, is administered at a dose as is described above either before, after, or simultaneously with the chlorambucil or R-chlorambucil administration. Hereby the CD37 antibody may be administered before, simultaneously with or after the CD20 antibody (e.g. day 1 CD37 mAb, day 2 CD20 mAb, days 3 and 4 chlorambucil; or day 1 CD20 mAb, days 2 and 3 chlorambucil, day 4 CD37 mAb; or day 1 CD20 mAb+CD37 mAb, days 2 and 3 chlorambucil). Simultaneously hereby means on the same day(s).

Furthermore, another preferred treatment cycle scheme for CLL or NHL comprises additional administration(s) of CD37 antibody in-between, for example in the middle of the treatment cycle at about 1-2 weeks or as another option once weekly.

The “therapeutically effective amount” of the antibody to be administered is the minimum amount necessary to prevent, ameliorate, or treat a disease or disorder.

CD37-positive malignancies include, without limitation, all malignancies that express CD37. B-cell malignancies belong to the group of CD37-positive malignancies. B-cell malignancies include, without limitation, B-cell lymphomas (e.g. various forms of Hodgkin's disease, B-cell non-Hodgkin's lymphoma (NHL) and related lymphomas (e.g. Waldenstrom's macroglobulinaemia (also called lymphoplasmacytic lymphoma or immunocytoma) or central nervous system lymphomas), leukemias (e.g. acute lymphoblastic leukemia (ALL), chronic lymphocytic leukemia (CLL; also termed B-cell chronic lymphocytic leukemia BCLL), hairy cell leukemia and chronic myelogenous leukemia). Additional B-cell malignancies include small lymphocytic lymphoma, B-cell prolymphocytic leukemia, lymphoplasmacytic lymphoma, splenic marginal zone lymphoma, plasma cell myeloma, solitary plasmacytoma of bone, extraosseous plasmacytoma, extra-nodal marginal zone B-cell lymphoma of mucosa-associated (MALT) lymphoid tissue, nodal marginal zone B-cell lymphoma, follicular lymphoma, mantle cell lymphoma, diffuse large B-cell lymphoma, mediastinal (thymic) large B-cell lymphoma, intravascular large B-cell lymphoma, primary effusion lymphoma, Burkitt's lymphoma/leukemia, grey zone lymphoma, B-cell proliferations of uncertain malignant potential, lymphomatoid granulomatosis, and post-transplant lymphoproliferative disorder. In addition, CD37-positive malignancies include, without limitation, T-cell lymphomas, is multiple myelomas, and acute lymphocytic leukemias.

The CD37 antibody may be administered alone or in combination with adjuvants that enhance the stability, facilitate administration of pharmaceutic compositions containing them in certain embodiments, provide increased dissolution or dispersion, increase activity, provide adjunct therapy, and the like. Advantageously, such combinations may utilize lower dosages of the active ingredient, thus reducing possible toxicity and adverse side effects.

### DESCRIPTION OF THE FIGURES

FIG. 1: DOHH2 TUMOR GROWTH KINETICS

DOHH2 tumor-bearing mice were treated with antibody A2, chlorambucil or with the combination of antibody A2 and chlorambucil. Median tumor volumes are plotted over time. Day 1 was the first day, day 16 the last day of the experiment. The symbols on the top denote the days on which treatment was given.

FIG. 2: WATER-FALL PLOT OF TUMOR VOLUME CHANGES ON DAY 16

DOHH2 tumor-bearing mice were treated with antibody A2, chlorambucil or with the combination of antibody A2 and chlorambucil. Individual changes from baseline at day 16 are plotted.

FIG. 3: CHANGE OF BODY WEIGHT OVER TIME

DOHH2 tumor-bearing mice were treated with antibody A2, chlorambucil or with the combination of antibody A2 and chlorambucil. Median changes of body weight are plotted over time. Day 1 was the first day, day 16 the last day of the experiment. The symbols on the top denote the days on which treatment was given.

### LEGEND TO SEQUENCE LISTING

SEQ ID NO 1: nucleic acid sequence variable heavy (Vh) chain

SEQ ID NO 2: amino acid sequence variable heavy chain

SEQ ID NO 3: nucleic acid sequence variable light (Vl) chain

SEQ ID NO 4: amino acid sequence variable light chain

SEQ ID NO 5: A2 heavy chain amino acid sequence

SEQ ID NO 6: A2 light chain amino acid sequence

SEQ ID NO 7: constant heavy chain amino acid sequence

SEQ ID NO 8: constant light chain amino acid sequence

SEQ ID NO 9: A4 heavy chain amino acid sequence

SEQ ID NO 10: A4 light chain amino acid sequence

SEQ ID NO 11: B2 heavy chain amino acid sequence

SEQ ID NO 12: B2 light chain amino acid sequence

SEQ ID NO 13: B4 heavy chain amino acid sequence

SEQ ID NO 14: B4 light chain amino acid sequence

SEQ ID NO 15: CDR1 heavy chain (H1)

SEQ ID NO 16: CDR2 heavy chain (H2)

SEQ ID NO 17: CDR3 heavy chain (H3)

SEQ ID NO 18: CDR1 light chain (L1)

SEQ ID NO 19: CDR2 light chain (L2)

SEQ ID NO 20: CDR3 light chain (L3)

SEQ ID NO 21: alternative CDR2 heavy chain (H2b)

### DETAILED DESCRIPTION OF THE INVENTION

The antibody A2 (=mAb A2) is a potent inducer of apoptosis both in the absence and presence of an IgG cross-linking antibody (see patent application WO2009/019312). We investigate here the efficacy of antibody A2 in combination with chlorambucil chemotherapy in a model of human follicular lymphoma (DOHH2) in C.B-17 scid mice.

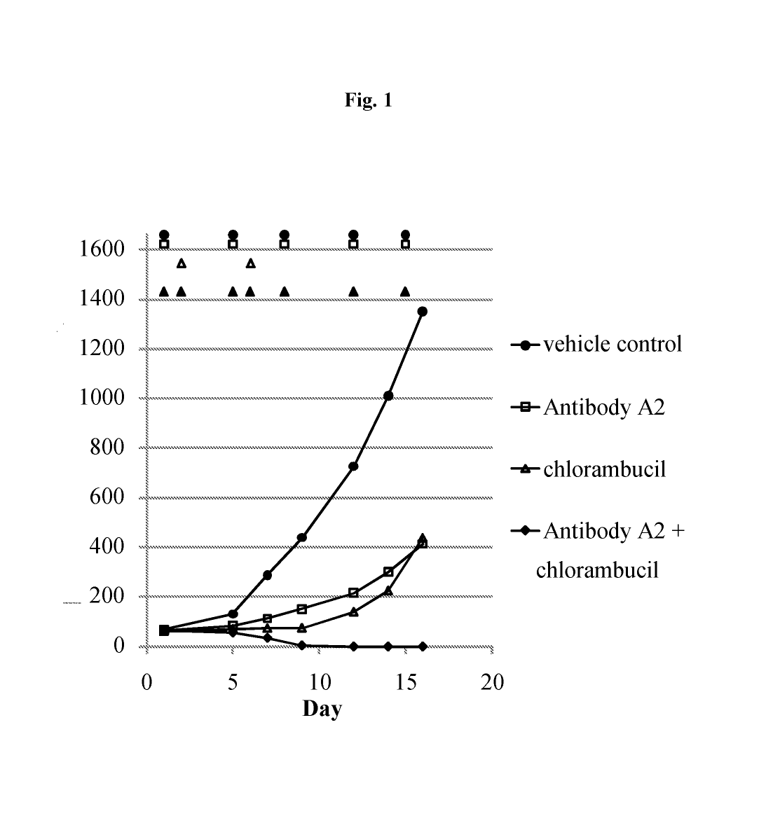

Two weeks of therapy with a combination of antibody A2 and chlorambucil was significantly more efficacious than single agent treatment with antibody A2 (median TGI=105% versus 73%, p=0.0014) or with chlorambucil (median TGI=105% versus 71%, p=0.0014)—FIG. 1 and Table 2 in Example 1. When the tumor volumes were analyzed based on descriptive statistics and by using a mixed model for repeated measurements (MMRM), combination therapy was found to show synergistic activity compared to the is corresponding monotherapies from day 9 on (p<0.0001).

Hence compared with single-agent treatment, the drug combination resulted in synergistic enhancement of efficacy.

An important finding from the studies reported in this patent application is the fact that mAb A2 exerts its pro-apoptotic activity without the need of an IgG cross-linking antibody, both as single agent and in combination with chlorambucil. IgG cross-linking in vitro is thought to mimic cross-linking by immune effector cells, e.g. NK cells, in vivo. Several antibodies described in the literature are dependent on IgG cross-linking to induce apoptosis, in particular the CD37-targeting antibody-like molecule CAS024 depends on IgG cross-linking (see European patent EP 2 132 228 B1). In cancer patients in vivo, the presence of immune effector cells may be limited or reduced, especially in patients treated with chemotherapeutic agents. Hence, an antibody which is able to induce apoptosis in the absence of an IgG cross-linking agent is considered favorable compared to an antibody which depends on IgG cross-linking, especially in combination with a chemotherapeutic agent which potentially impairs immune effector cell activity. A2 is such an antibody which in combination with chlorambucil is able to induce surprisingly more than additive apoptosis than either agent alone without the need for IgG cross-linking, which is considered advantageous for the treatment of cancer patients.

### DEFINITIONS

Terms not specifically defined herein should be given the meanings that would be given to them by one of skill in the art in light of the disclosure and the context. The general embodiments “comprising” or “comprised” encompass the more specific embodiment “consisting of”. Furthermore, singular and plural forms are not used in a limiting way. As used in the specification, however, unless specified to the contrary, the following terms have the meaning indicated and the following conventions are adhered to.

The term “chlorambucil” describes a chemotherapeutic agent. Chlorambucil (marketed as Leukeran) is a nitrogen mustard alkylating agent used in the treatment of hematologic malignancies, e.g. chronic lymphocytic leukemias and lymphomas. It belongs to the is family of drugs called alkylating agents, which are widely used for the treatment of malignant neoplasms (cancer). The chemical mass formula is C₁₄C₁₉Cl₂NO₂ with a molecular mass of 304.212 g/mol. The systematic (IUPAC) name is 4-[Bis(2-chloroethy)amino]benzenebutaonic acid. The chemical structure of chlorambucil is as follows:

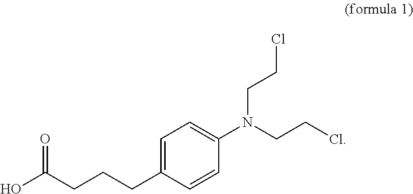 [SMILES: O=C(O)CCCc1ccc(N(CCCl)CCCl)cc1]

“Rituximab” is a chimeric monoclonal antibody against the protein CD20. The chemical mass formula of Rituximab is C₆₄₁₆H₉₈₇₄N₁₆₈₈O₁₉₈₇S₄₄ with a molecular mass of 143859.7 g/mol.

“CD37”, a member of the tetraspanin superfamily, is a heavily glycosylated cell surface molecule with four transmembrane domains and two extracellular loops. CD37 is predominantly expressed on B-cells and B-cell malignancies, low level expression of CD37 has been reported on T-cells, granulocytes, and monocytes. High levels of CD37 expression have been observed in samples of patients with chronic lymphocytic leukemia (CLL) and different subtypes of non-Hodgkin's lymphoma (NHL) including mantle cell lymphoma (MCL) (Schwartz-Albiez et al, Journal Immunol 140: 905-914, 1988; Barrena et al., Leukemia 19: 1376-1383, 2005). This expression pattern makes CD37 an attractive target for antibody-mediated cancer therapy. Binding of a CD37-specific mAb to cancer cells may trigger various mechanisms of action: First, after the antibody binds to the extracellular domain of the CD37 antigen, it may activate the complement cascade and lyse the targeted cell. Second, an anti-CD37 antibody may mediate antibody-dependent cell-mediated cytotoxicity (ADCC) to the target cell, which occurs after the Fc portion of the bound antibody is recognized by appropriate receptors on cytotoxic cells of the immune system. Third, the antibody may alter the ability of B-cells to respond to antigen or other stimuli. Finally, anti-CD37 antibody may initiate programmed cell death (apoptosis).

“CD37 positive”, “CD37 positive cells” or “CD37 positive malignancies” means that the detection of CD37 is possible/feasible by immunohistochemistry, flow cytometry such as FACS (fluorescence activated cell sorter) analysis (of e.g. blood, bone marrow or cell suspensions) or alternative techniques. Suitable assays to detect CD37 positive cells/malignancies are well known to a person skilled in the art.

The terms “CD37 antibody”, “CD37 antibody molecule”, “anti-CD37 antibody” and “anti-CD37 antibody molecule” as used in the present invention specifically relate to an antibody with a binding specificity for CD37 antigen. Examples of such antibodies are known in the art and are further described below.

The terms “anti-CD37 antibody molecule”, “anti-CD37 antibody”, “CD37 antibody” and “CD37 antibody molecule” are used interchangeably.

The term “CD37 antibody” or “anti-CD37 antibody molecule” encompasses anti-CD37 antibodies and anti-CD37 antibody fragments as well as conjugates with antibody molecules. Antibodies include, in the meaning of the present invention, chimeric monoclonal and humanized monoclonal antibodies. The term “antibody”, which may interchangeably be used with “antibody molecule”, shall encompass complete immunoglobulins (as they are produced by lymphocytes and for example present in blood sera), monoclonal antibodies secreted by hybridoma cell lines, polypeptides produced by recombinant expression in host cells, which have the binding specificity of immunoglobulins or monoclonal antibodies, and molecules which have been derived from such antibodies by modification or further processing while retaining their binding specificity.

In certain embodiments, the antibody molecule of the invention is a chimeric CD37-specific antibody that has the heavy chain variable region of a non-human antibody defined is in a) or b) fused to the human heavy chain constant region IgG1 and the light chain variable region of a non-human antibody defined in a) or b) fused to the human light chain constant region kappa.

The CD37 antibody may also be in the form of a conjugate, i.e. an antibody molecule that is chemically coupled to a cytotoxic agent, particularly a cytotoxic agent that induces cytotoxicity (e.g. apoptosis or mitotic arrest) of tumor cells. As a result of normal pharmacologic clearance mechanisms, an antibody employed in a drug conjugate (an “immunoconjugate”) contacts and binds to target cells only in limited amounts. Therefore, the cytotoxic agent employed in the conjugate must be highly cytotoxic such that sufficient cell killing occurs to elicit a therapeutic effect. As described in US 2004/0241174, examples of such cytotoxic agents include taxanes (see, e.g. WO 01/38318 and WO 03/097625), DNA-alkylating agents (e.g., CC-1065 analogs), anthracyclines, tubulysin analogs, duocarmycin analogs, doxorubicin, auristatin E, ricin A toxin, and cytotoxic agents comprising a reactive polyethylene glycol moiety (see, e.g., Sasse et al., 2000; Suzawa et al., 2000; Ichimura et al., 1991; Francisco et al., 2003; U.S. Pat No. 5,475,092; U.S. Pat No. 6,340,701; U.S. Pat. No. 6,372,738; and U.S. 6,436,931; US 2001/0036923; US 2004/0001838; US 2003/0199519; and WO 01/49698).

In a preferred embodiment, the cytotoxic agent is a maytansinoid, i.e. a derivative of maytansine (CAS 35846538), maytansinoids being known in the art to include maytansine, maytansinol, C-3 esters of maytansinol, and other maytansinol analogues and derivatives (see, e.g., U.S. Pat. No. 5,208,020; and U.S. Pat. No. 6,441,163).

Anti-CD37 antibody immunoconjugates may be designed and synthesized as described in WO 2007/077173 for anti-FAP immunoconjugates.

In a further embodiment, the anti-CD37 molecule of the invention may be radioactively labelled to form a radioimmunoconjugate, an approach suggested for the anti-CD37 antibody MB-1 (Buchsbaum et al., 1992, see above). Radionuclides with advantageous radiation properties are known in the art, examples are Phosphorus-32, Strontium-89, Yttrium-90, Iodine-131, Samarium-153, Erbium-169, Ytterbium-175, Rhenium-188, that have been successfully and stably coupled to MAbs. The CD37 antibodies of the invention is may be labelled with various radionuclides using direct labelling or indirect labelling methods known in the art, as described in U.S. Pat. No. 6,241,961. A review on technologies for generating and applying novel radiolabeled antibody conjugates that are useful in the present invention, is given by Goldenberg and Sharkey, 2007.

An antibody molecule of the invention, whether Fc-engineered or not, may also be bispecific, i.e. an antibody molecule that binds to two different targets, one of them being CD37, the other one being selected from e.g. surface antigens expressed by T cells, e.g. CD3, CD16 and CD56.

The term “antibody” or “antibodies” comprises monoclonal, polyclonal, multispecific and single chain antibodies and fragments thereof such as for example Fab, Fab′, F(ab′)₂, Fc and Fc′ fragments, light (L) and heavy (H) immunoglobulin chains and the constant, variable or hypervariable regions thereof as well as Fv and Fd fragments. The term “antibody” or “antibodies” comprises antibodies of human or non-human origin, humanised as well as chimeric antibodies and furthermore Fc-engineered antibodies or Fc-fusion molecules.

Fab fragments (fragment antigen binding=Fab) consist of the variable regions of both chains which are held together by the adjacent constant regions. They may be produced for example from conventional antibodies by treating with a protease such as papain or by DNA cloning. Other antibody fragments are F(ab′)₂ fragments which can be produced by proteolytic digestion with pepsin.

By gene cloning it is also possible to prepare shortened antibody fragments which consist only of the variable regions of the heavy (VH) and light chain (VL). These are known as Fv fragments (fragment variable=fragment of the variable part). As covalent binding via the cysteine groups of the constant chains is not possible in these Fv fragments, they are often stabilised by some other method. For this purpose the variable regions of the heavy and light chains are often joined together by means of a short peptide fragment of about 10 to 30 amino acids, preferably 15 amino acids. This produces a single polypeptide chain in which VH and VL are joined together by a peptide linker Such antibody fragments are is also referred to as single chain Fv fragments (scFv). Examples of scFv antibodies are known in the art.

In past years various strategies have been developed for producing multimeric scFv derivatives. The intention is to produce recombinant antibodies with improved pharmacokinetic properties and increased binding avidity. In order to achieve the multimerisation of the scFv fragments they are produced as fusion proteins with multimerisation domains. The multimerisation domains may be, for example, the CH3 region of an IgG or helix structures (“coiled coil structures”) such as the Leucine Zipper domains. In other strategies the interactions between the VH and VL regions of the scFv fragment are used for multimerisation (e.g. dia, tri- and pentabodies).

The term “diabody” is used in the art to denote a bivalent homodimeric scFv derivative. Shortening the peptide linker in the scFv molecule to 5 to 10 amino acids results in the formation of homodimers by superimposing VH/VL chains. The diabodies may additionally be stabilised by inserted disulphite bridges. Examples of diabodies can be found in the literature.

The term “minibody” is used in the art to denote a bivalent homodimeric scFv derivative. It consists of a fusion protein which contains the CH3 region of an immunoglobulin, preferably IgG, most preferably IgG1, as dimerisation region. This connects the scFv fragments by means of a hinge region, also of IgG, and a linker region. Examples of such s minibodies are known in the art.

The term “triabody” is used in the art to denote a trivalent homotrimeric scFv derivative. The direct fusion of VH-VL without the use of a linker sequence leads to the formation of trimers.

The fragments known in the art as mini antibodies which have a bi, tri- or tetravalent structure are also derivatives of scFv fragments. The multimerisation is achieved by means of di-, tri- or tetrameric coiled coil structures.

is There are also “scaffold proteins” or “scaffold antibodies” known in the art. Using this term, a scaffold protein means any functional domain of a protein, especially an antibody, that is coupled by genetic cloning or by co-translational processes with another protein or part of a protein that has another function.

The term “Complementary determining region” or “CDR” or “CDRs” of an antibody/antibody molecule means the hypervariable regions (also called Complementarity Determining Regions, abbreviated to “CDRs”) of immunoglobulins. The CDRs were originally defined by Kabat et al., (“Sequences of Proteins of Immunological Interest” Kabat, E., of al., U.S. Department of Health and Human Services, (1983) and Kabat E. A., Wu T. T., Perry H. M., Gottesman K. S. and Foeller C. Sequences of Proteins of Immunological Interest (5th Ed.). NIH Publication No. 91-3242. U.S. Department of Health and Human Services, Public Health Service, National Institutes of Health, Bethesda, Md. 1991) based on extent of sequence variability of numerous antibody sequences. The CDRs are believed to contact the target antigen of an antibody and to be primarily responsible for binding. Chothia et al (Chothia and Lesk, J. Mol. Biol., 196:901-917 (1987)) have given an alternate definition of the hypervariable regions or CDRs. The Chothia definition is based on the residues that constitute the loops in the 3-dimensional structures of antibodies.

In the specific context of the present invention the CDRs are determined on the basis of the Kabat system. From the sequences of the variable regions as shown in SEQ ID NO:2 and SEQ ID NO:4, the CDR sequence can be routinely determined by searching the Kabat sequence database for sequence features. The 3 CDRs contained within the variable heavy chain as shown in SEQ ID NO:2 comprise preferably positions 31-35 (H1, SEQ ID NO: 15), 50-66 (H2, SEQ ID NO: 16) or 50-62 (H2b, SEQ ID NO: 21) and 99-105 (H3, SEQ ID NO: 17), the 3 CDRs contained within the variable light chain as shown in SEQ ID NO:4 comprise preferably positions 24-34 (L1, SEQ ID NO: 18), 50-56 (L2, SEQ ID NO: 19) and 89-97 (L3, SEQ ID NO: 20).

The term “treatment cycle” describes a time period of between 1 to 8 weeks, preferably 3 to 6 weeks, also preferably 3 to 4 weeks, most preferably 4 weeks, wherein the patient is receives at least one dose of the CD37 antibody and at least one dose of chlorambucil.

The terms “dose” and “dosage” are used interchangeably.

The terms “NHL” and “B-NHL” are used interchangeably.

### Embodiments

The present invention concerns a CD37 antibody for use in a method for the treatment of a patient suffering from a CD37-positive malignancy, preferably a B-cell malignancy, most preferably chronic lymphocytic leukemia (CLL) or B-cell non-Hodgkin's lymphoma (B-NHL), in combination with chlorambucil, whereby the CD37 antibody comprises:
a variable heavy chain comprising CDRs have the SEQ ID NOs: 15, 16 or 21, and 17, and
a variable light chain comprising CDRs having the SEQ ID NOs: 18, 19 and 20.

The present invention further concerns a CD37 antibody for use in a method for the treatment of a patient suffering from a CD37-positive malignancy, preferably a B-cell malignancy, most preferably chronic lymphocytic leukemia (CLL) or B-cell non-Hodgkin's lymphoma (B-NHL), in combination with chlorambucil and a CD20 antibody like Rituximab (called R-chlorambucil), whereby the CD37 antibody comprises:
a variable heavy chain comprising CDRs have the SEQ ID NOs: 15, 16 or 21, and 17, and
a variable light chain comprising CDRs having the SEQ ID NOs: 18, 19 and 20.

The present invention furthermore concerns a CD37 antibody for use in a method for the treatment of a patient suffering from a CD37-positive malignancy, preferably a B-cell malignancy, most preferably chronic lymphocytic leukemia (CLL) or B-cell non-Hodgkin's lymphoma (B-NHL), in combination with a chemotherapeutic agent (such as e.g. an alkylating agent, such as e.g. chlorambucil) and a CD20 antibody like Rituximab (called R-chemotherapy), whereby the CD37 antibody comprises:
a variable heavy chain comprising CDRs having the SEQ ID NOs: 15, 16 or 21, and 17, and
a variable light chain comprising CDRs having the SEQ ID NOs: 18, 19 and 20.

In a specific embodiment the CD37 antibody is a chimeric antibody. Preferably said chimeric antibody comprises the human constant heavy chain amino acid sequence SEQ ID NO:7 and the human constant light chain amino acid sequence SEQ ID NO:8.

In a preferred embodiment the CD37 antibody comprises the heavy chain amino acid sequence SEQ ID NO: 5 and the light chain amino acid sequence SEQ ID NO: 6 (=>A2). In a specific embodiment the CD37 antibody is a humanized antibody. Preferably said humanized CD37 antibody comprises the heavy chain amino acid sequence SEQ ID NO: 11 and the light chain amino acid sequence 12 (=>B2).

In a specific embodiment the patient receives at least one dose of the CD37 antibody and at least one dose of chlorambucil during a treatment cycle, whereby a treatment cycle is a time period of about 1 to 6 weeks, preferably 3 to 6 weeks, preferably 3 to 4 weeks, most preferably 4 weeks.

In another specific embodiment the patient additionally receives at least one dose of a CD20 antibody such as Rituximab.

In a further specific embodiment the CD37 antibody is administered to said patient simultaneously with the administration of chlorambucil.

In another embodiment the CD37 antibody is administered to said patient after the administration of chlorambucil, preferably within 24 hrs or within 36 hrs after the administration of chlorambucil.

In a further embodiment the CD37 antibody is administered to said patient before the administration of chlorambucil, preferably within 24 hrs or within 36 hrs before the administration of chlorambucil.

In another preferred embodiment the CD37 antibody is administered to said patient after a 2 day consecutive application of chlorambucil, preferably within 24 hrs or within 36 hrs after the administration of the second chlorambucil dosage. In another preferred is embodiment the CD37 antibody is administered to said patient the day after a 2 day consecutive application of chlorambucil, whereby the day after preferably means within 24 hrs or within 36 hrs after the administration of chlorambucil. Preferably chlorambucil is administered to said patient on days 1 and 2 of a 1 to 6 week treatment cycle, more preferably of a 3 to 6 week treatment cycle, preferably of a 3-4 week treatment cycle, most preferably of a 4 week treatment cycle, and the CD37 antibody is administered on day 3 of the treatment cycle.

In a further preferred embodiment the CD37 antibody is administered to said patient before a 2 day consecutive application of chlorambucil, preferably within 24 hrs or within 36 hrs before the administration of the first chlorambucil dosage. In another preferred embodiment the CD37 antibody is administered to said patient the day before a 2 day consecutive application of chlorambucil, whereby the day before preferably means within 24 hrs or within 36 hrs before the administration of chlorambucil. Preferably chlorambucil is administered to said patient on days 2 and 3 of a 1 to 6 week treatment cycle, preferably a 3 to 6 week treatment cycle, more preferably of a 3-4 week treatment cycle, most preferably of a 4 week treatment cycle, and the CD37 antibody is administered on day 1 of the treatment cycle.

In a specific embodiment the CD37 antibody is additionally administered at least one more time in between, preferably in the middle of the treatment cycle at about 2 weeks.

In another embodiment the CD37 antibody is additionally administered at least one more time during a treatment cycle, preferably in the middle of the treatment cycle at about 2 weeks or once weekly, whereby the treatment cycle is a time period of between 1 to 6 weeks, preferably 3 to 6 weeks, preferably 3 to 4 weeks, most preferably 4 weeks. The treatment cycle is a time period of between 1 to 6 weeks, preferably 3 to 6 weeks, preferably 3 to 4 weeks, most preferably 4 weeks, wherein the patient receives at least one dose of the CD37 antibody and at least one dose of chlorambucil.

The CD37 antibody, preferably A2 (CD37 antibody comprising SEQ ID Nos: 5 and 6) and B2 (CD37 antibody comprising SEQ ID Nos: 11 and 12), most preferably A2, is is administered in a dose of about 0.01 μg/kg to 40 mg/kg or in a dose of about 10μg/kg to 40 mg/kg or in a dose of about 1 mg and 2800 mg per patient. Administration to the patient may occur by one or more separate administrations. It may occur for example by infusion such as continuous infusion.

For A2 (CD37 antibody comprising SEQ ID Nos:5 and 6), the estimated weekly dose for a 70 kg human is in the range of 1 mg to 2800 mg, preferably 1mg to 400 mg weekly or 2 mg to 800 mg every 2 weeks. The estimated human weekly dose of B2 (CD37 antibody comprising SEQ ID Nos:11 and 12) for a 70 kg human is in the range of 1 mg to 2800 mg, preferably in the range of 1 mg to 1000 mg, e.g. 100 mg to 385 mg weekly or 200 mg to 770 mg every two weeks for a 70 kg person.

Chlorambucil is preferably dosed on two consecutive days (e.g. d1−d2) of a treatment cycle, which is preferably 3-4 weeks (=21-28 days) long. The dose for chlorambucil ranges between 50-150 mg/m2 body surface on 2 treatment days of a 3 to 4 week long treatment cycle. Preferably the dose of chlorambucil ranges between 70-120 mg/m² body surface or between 100-150 mg/m² body surface on d1+d2 of a treatment cycle.

In a further embodiment chlorambucil is preferably dosed daily of a treatment cycle, which is preferably 3-6 weeks (=21-36 days) long. The dose for chlorambucil ranges between 0.0.5-0.4 mg/kg. Preferably the dose ranges between 0.1-0.3 mg/kg. Alternate schedules for the treatment of CLL employing intermittent, biweekly, or once-monthly pulse doses of chlorambucil can be used. Intermittent schedules of chlorambucil begin with an initial single dose of 0.4 mg/kg.

For the treatment of a CLL patient chlorambucil is preferably administered at a dosage of 100 mg/m² body surface on days 1 and 2 of the treatment cycle, which is preferably 3-4 to weeks long, most preferably 4 weeks.

Alternatively for the treatment of a CLL patient chlorambucil is preferably administered at a dosage of 0.1 to 0.2 mg/kg daily in a treatment cycle, which is preferably 3-6 weeks long, most preferably 4 weeks.

For the treatment of a NHL patient chlorambucil is preferably administered at a dosage of 120 mg/m² body surface on days 1 and 2 of the treatment cycle, which is preferably 3-4 weeks long, most preferably 3 weeks.

Alternatively for the treatment of a NHL patient chlorambucil is preferably administered at a dosage of 0.1 to 0.2 mg/kg daily in a treatment cycle, which is preferably 3-6 weeks long, most preferably 4 weeks.

Furthermore preferred is a chlorambucil dose in the range of 60-70 mg/m² body surface on d1+d2 of a treatment cycle.

In a further specific embodiment chlorambucil is administered as a one-time administration per treatment cycle preferably with a dose of 70-400 mg/m² body surface.

The chlorambucil dose as described above is administered preferably on day 1 and on day 2 of a 3-4 week treatment cycle. Furthermore, preferred is the administration of a chlorambucil dose as described above on 2 consecutive days following a CD37 antibody administration (e.g. day1=CD37 administration in any of the dosages as described above, days 2+3=chlorambucil administration in any of the dosages as described above) of a preferably 3-4 week long treatment cycle.

The chlorambucil dose may be administered by any way, e.g. infusion, parenteral or oral administration. Preferably the dose range for oral administration of chlorambucil ranges from 10 to 1000 mg, more preferably 25 to 600 mg or 50 to 200 mg, most preferably about 100 mg.

The CD37 antibody dose may be administered by any way, e.g. infusion such as continuous infusion, subcutaneous injection, inhalation, parenteral or oral administration.

In a specific embodiment of the present in invention a CD37 antibody is administered in combination with chlorambucil as first line treatment. First line treatment means as a first is treatment option (before other treatment options are performed/used). In a preferred embodiment of the present in invention a CD37 antibody is administered in combination with chlorambucil as second line treatment of CLL.

In another specific embodiment of the present in invention a CD37 antibody is administered in combination with chlorambucil as second line or third or fourth or further line treatment. Second, third, fourth or further line treatment means the administration as a second, third, fourth or later/further line treatment option after one or more other treatment(s) already has (have) been performed/used.

For the treatment of a patient suffering from CLL a preferred treatment cycle scheme lasts for a time period of 4 weeks, whereby chlorambucil is preferably administered at a dose of 100 mg/m² body surface preferably on day 1 and 2 and whereby at least one CD37 antibody, preferably A2 or B2, is administered at a dose as described above either before, after or simultaneously with the chlorambucil administration. Simultaneously hereby means on the same day. Simultaneously furthermore may mean within six hours of each other or within one hour of each other or with the same injection. Furthermore, another preferred treatment cycle scheme for CLL comprises additional administration(s) of CD37 antibody in-between, for example in the middle of the treatment cycle at about 2 weeks.

For the treatment of a patient suffering from NHL a preferred treatment cycle scheme lasts for a time period of 3 weeks, whereby chlorambucil is preferably administered at a dose of 120 mg/m2 body surface preferably on day 1 and 2 and whereby at least one CD37 antibody, preferably A2 or B2, is administered at a dose as described above either before, after or simultaneously with the chlorambucil administration. Simultaneously hereby means on the same day. Simultaneously furthermore may mean within six hours of each other or within one hour of each other or with the same injection. Furthermore, another preferred treatment cycle scheme for NHL comprises additional administration(s) of CD37 antibody in-between, for example once a week, thus resulting in several, preferably 3 to 4, most preferably 4 administrations of CD37 antibody per treatment cycle.

In a specific embodiment of the present invention any of the described treatment cycle schemes for chlorambucil as described in the paragraphs above is combined with the administration of an antibody targeting CD20 such as Rituximab. This treatment option is referred to as R-chlorambucil.

In a preferred embodiment of the present invention any of the described treatment cycle schemes for chlorambucil+CD37 mAb as described in the paragraphs above is combined with the administration of an antibody targeting CD20 such as Rituximab. This treatment option is referred to as R-chlorambucil+CD37 mAb.

In a preferred treatment cycle scheme for R-chlorambucil the Rituximab (or alternatively any other antibody targeting CD20) is embedded into the chlorambucil treatment cycle and dosing scheme (dosing as described in the paragraphs above), preferably by administering the antibody targeting CD20 (e.g. Rituximab) simultaneously with chlorambucil on the 1^{st} treatment day or by administering the antibody targeting CD20 (e.g. Rituximab) before the first chlorambucil administration (e.g. day 1 Rituximab, days 2 and 3 chlorambucil). A preferred dose for Rituximab is 100-500 mg/m² body surface, preferably 375-500 mg/m², most preferably 375 mg/m².

For CD37 combination therapy during a R-chlorambucil treatment cycle at least one CD37 antibody, preferably A2 or B2, is administered at a dose as described above either before, after, or simultaneously with the R-chlorambucil administration. Simultaneously hereby means on the same day(s). Furthermore, another preferred treatment cycle scheme for NHL comprises additional administration(s) of CD37 antibody in-between, for example in the middle of the treatment cycle at about 1-2 weeks, preferably 1.5 weeks.

The present invention further concerns a method of reducing CD37-positive cells, more specifically B-cells, comprising exposing B-cells to a combination of a CD37 antibody and chlorambucil or R-chlorambucil, whereby said CD37 antibody comprises:
a) a variable heavy chain comprising CDRs have the SEQ ID NOs: 15, 16 or 21, and 17, and
b) a variable light chain comprising CDRs having the SEQ ID NOs: 18, 19 and 20.

The present invention further concerns a method of reducing CD37-positive cells, more specifically B-cells, comprising exposing B-cells to a combination of a CD37 antibody, a chemotherapeutic agent such as e.g. an alkylating agent and a CD20 antibody such as Rituximab, whereby said CD37 antibody comprises:
a) a variable heavy chain comprising CDRs have the SEQ ID NOs: 15, 16 or 21, and 17, and
b) a variable light chain comprising CDRs having the SEQ ID NOs: 18, 19 and 20.

The present invention furthermore concerns a method of depleting CD37 expressing B-cells from a population of cells comprising administering to said population of cells: a) a CD37 antibody or a pharmaceutical composition comprising a CD37 antibody and b) chlorambucil or R-chlorambucil, wherein said method is preferably carried out in vitro, and whereby said CD37 antibody comprises:
a) a variable heavy chain comprising CDRs have the SEQ ID NOs: 15, 16 or 21, and 17, and
b) a variable light chain comprising CDRs having the SEQ ID NOs: 18, 19 and 20.

The present invention further concerns a method of reducing CD37-positive cells comprising:
a) Exposing CD37-positive cells to a CD37 antibody and
b) Exposing CD37-positive cells to chlorambucil or R-chlorambucil, whereby said CD37 antibody of step a) comprises:
i) a variable heavy chain comprising CDRs have the SEQ ID NOs: 15, 16 or 21, and 17, and
ii) a variable light chain comprising CDRs having the SEQ ID NOs: 18, 19 and 20.

The present invention additionally concerns a method of reducing CD37-positive cells comprising:
a) Exposing CD37-positive cells to a CD37 antibody, and
b) Exposing CD37-positive cells to a chemotherapeutic agent such as e.g. an alkylating agent, and
c) Exposing CD37-positive cells to a CD20 antibody such as Rituximab, whereby said CD37 antibody of step a) comprises:
i) a variable heavy chain comprising CDRs have the SEQ ID NOs: 15, 16 or 21, and 17, and
ii) a variable light chain comprising CDRs having the SEQ ID NOs: 18, 19 and 20.

The present invention furthermore concerns a method of reducing B-cells comprising:
a) Exposing B-cells to a CD37 antibody and
b) Exposing B-cells to chlorambucil or R-chlorambucil, whereby said CD37 antibody of step a) comprises:
i) a variable heavy chain comprising CDRs have the SEQ ID NOs: 15, 16 or 21, and 17, and
ii) a variable light chain comprising CDRs having the SEQ ID NOs: 18, 19 and 20.

In a specific embodiment the CD37 antibody is a chimeric antibody. Preferably said chimeric antibody comprises the human constant heavy chain amino acid sequence SEQ ID NO:7 and the human constant light chain amino acid sequence SEQ ID NO:8.

In a preferred embodiment the CD37 antibody comprises the heavy chain amino acid sequence SEQ ID NO: 5 and the light chain amino acid sequence SEQ ID NO: 6 (=>A2).

In a specific embodiment the CD37 antibody is a humanized antibody. Preferably said humanized CD37 antibody comprises the heavy chain amino acid sequence SEQ ID NO: 11 and the light chain amino acid sequence 12 (=>B2).

In a specific embodiment of any of said methods the CD37-positive cells are exposed to the CD37 antibody and chlorambucil simultaneously. Said CD37-positive cells are preferably B-cells.

In another embodiment of any of said methods the CD37-positive cells are exposed to the CD37 antibody after they are exposed to chlorambucil, preferably within 24 hrs or within 36 hrs after they are exposed to chlorambucil. Said CD37-positive cells are preferably B-cells.

In a further embodiment of any of said methods the CD37-positive cells are exposed to the CD37 antibody before they are exposed to chlorambucil, preferably within 24 hrs or within 36 hrs before they are exposed to chlorambucil. Said CD37-positive cells are preferably B-cells.

In a specific embodiment said method is carried out in vivo.

In a specific embodiment said method is carried out in vitro.

The present invention further concerns a kit for reducing CD37-positive cells comprising:
a) a container comprising a CD37 antibody, whereby said CD37 antibody comprises:
   i) a variable heavy chain comprising CDRs having the SEQ ID NOs: 15, 16 or 21, and 17, and
   ii) a variable light chain comprising CDRs having the SEQ ID NOs: 18, 19 and 20, and
b) a protocol for using the kit to reduce CD37-positive cells (by administration of the CD37 antibody of step a) in combination with a chemotherapeutic agent/treatment such as chlorambucil or R-chlorambucil. Said CD37-positive cells are preferably B-cells.

The present invention specifically concerns a kit for reducing CD37-positive cells comprising:
a) container comprising a CD37 antibody, whereby said CD37 antibody comprises:
   i) a variable heavy chain comprising CDRs having the SEQ ID NOs: 15, 16 or 21, and 17, and
   ii) a variable light chain comprising CDRs having the SEQ ID NOs: 18, 19 and 20, and
b) protocol for using the kit to reduce CD37-positive cells by administration of the CD37 antibody of step a) in combination with chlorambucil and/or
c) optionally a protocol for using the kit to reduce CD37-positive cells by administration of the CD37 antibody of step a) in combination with chlorambucil is and a CD20 antibody such as Rituximab.

The present invention furthermore concerns a kit for reducing CD37-positive cells comprising: a) first container comprising a CD37 antibody, whereby said CD37 antibody comprises:
i) a variable heavy chain comprising CDRs have the SEQ ID NOs: 15, 16 or 21, and 17, and
ii) a variable light chain comprising CDRs having the SEQ ID NOs: 18, 19 and 20, and

b) second container comprising a chemotherapeutic agent/treatment such as chlorambucil, and

c) optionally a third container comprising a CD20 antibody like Rituximab, and

d) a protocol for using the kit to reduce CD37-positive cells. Said CD37-positive cells are preferably B-cells.

In a specific embodiment the protocol in step c) indicates to administer the CD37 antibody and chlorambucil or R-chlorambucil simultaneously.

In another embodiment the protocol in step c) indicates to administer the CD37 antibody before chlorambucil or R-chlorambucil, preferably within 24 hrs or within 36 hrs before the administration of chlorambucil or R-chlorambucil.

In a further embodiment the protocol in step c) indicates to administer the CD37 antibody after chlorambucil or R-chlorambucil, preferably within 24 hrs or within 36 hrs after the administration of chlorambucil or R-chlorambucil.

In a specific embodiment the protocol in step c) indicates to administer the kit components to a patient suffering from a CD37-positive malignancy, preferably a B-cell malignancy, preferably chronic lymphocytic leukemia (CLL) or NHL, most preferably CLL.

In a further specific embodiment the protocol in step c) indicates that the patient receives at least one dose of the CD37 antibody and at least one dose of chlorambucil or R-chlorambucil during a treatment cycle, whereby a treatment cycle is a time period of about is 1 to 6 weeks, preferably 3 to 4 weeks, most preferably 4 weeks.

In further specific embodiments the protocol in step c) indicates treatment cycles and/or dosage schemes as described above for the second medical use of the described CD37 antibodies.

The present invention further concerns an article of manufacture comprising a CD37 antibody and a chemotherapeutic agent/treatment such as chlorambucil or R-chlorambucil and a label indicating a method as described above, whereby the CD37 antibody comprises: a) a variable heavy chain comprising CDRs have the SEQ ID NOs: 15, 16 or 21, and 17, and b) a variable light chain comprising CDRs having the SEQ ID NOs: 18, 19 and 20.

The present invention furthermore concerns a pharmaceutical composition comprising, a CD37 antibody, chlorambucil or R-chlorambucil, and a pharmaceutically acceptable carrier,

whereby the CD37 antibody comprises:

a) a variable heavy chain comprising CDRs have the SEQ ID NOs: 15, 16 or 21, and 17, and

b) a variable light chain comprising CDRs having the SEQ ID NOs: 18, 19 and 20.

In a specific embodiment the pharmaceutical composition comprises as the active ingredient a CD37 antibody and chlorambucil, and additionally a pharmaceutically acceptable carrier,

whereby the CD37 antibody comprises:

a) The CDRs contained within the variable heavy chain as shown in SEQ ID NO:2, preferably said CDRs have SEQ ID NOs: 15, 16 or 21, and 17, and

b) The CDRs contained within the variable light chain as shown in SEQ ID NO:4, preferably said CDRs have SEQ ID NOs: 18, 19 and 20.

The present invention further concerns the pharmaceutical composition as described above is for use as a medicament.

The present invention furthermore concerns the pharmaceutical composition as described above for use in a method for the treatment of a patient suffering from a B-cell malignancy, preferably for use in a method for the treatment of a chronic lymphocytic leukemia (CLL) patient.

The present invention further concerns a method of treating a B-cell malignancy comprising administering a therapeutically effective amount of a CD37 antibody in combination with a chemotherapeutic agent/treatment such as chlorambucil or R-chlorambucil to a patient in need thereof, whereby the CD37 antibody comprises:

a) a variable heavy chain comprising CDRs have the SEQ ID NOs: 15, 16 or 21, and 17, and

b) a variable light chain comprising CDRs having the SEQ ID NOs: 18, 19 and 20.

The present invention additionally concerns a method of treating a B-cell malignancy comprising administering a therapeutically effective amount of a CD37 antibody in combination with a chemotherapeutic agent/treatment and a CD20 antibody like Rituximab to a patient in need thereof, whereby the CD37 antibody comprises:

a) a variable heavy chain comprising CDRs have the SEQ ID NOs: 15, 16 or 21, and 17, and

b) a variable light chain comprising CDRs having the SEQ ID NOs: 18, 19 and 20.

The present invention furthermore concerns a method for treating a patient suffering from a B-cell malignancy selected from B-cell non-Hodgkin's lymphoma, B-cell chronic lymphocytic leukemia and multiple myeloma, comprising administering to said patient an effective amount of a pharmaceutical composition of the present invention.

The present invention further concerns a method of treating a B-cell malignancy comprising administrating a therapeutically effective amount of a) A CD37 antibody and b) chlorambucil or R-chlorambucil, to a patient in need thereof, whereby the CD37 is antibody comprises:

a) a variable heavy chain comprising CDRs have the SEQ ID NOs: 15, 16 or 21, and 17, and

b) a variable light chain comprising CDRs having the SEQ ID NOs: 18, 19 and 20.

In a specific embodiment of said methods of treatment the patient receives at least one dose of the CD37 antibody and at least one dose of chlorambucil or R-chlorambucil during a treatment cycle, whereby a treatment cycle is a time period of about 1 to 6 weeks, preferably 3 to 4 weeks, most preferably 4 weeks.

In a specific embodiment of any of said methods the B-cells are exposed to the CD37 antibody and chlorambucil or R-chlorambucil simultaneously.

In another embodiment of any of said methods the B-cells are exposed to the CD37 antibody after they are exposed to chlorambucil or R-chlorambucil, preferably within 24 hrs or within 36 hrs after they are exposed to chlorambucil or R-chlorambucil.

In a further embodiment of any of said methods the B-cells are exposed to the CD37 antibody before they are exposed to chlorambucil or R-chlorambucil, preferably within 24 hrs or within 36 hrs before they are exposed to chlorambucil or R-chlorambucil.

In a specific embodiment said method is carried out in vivo.

In a specific embodiment said method is carried out in vitro.

The dosage regimens described above for the second medical use of CD37 antibodies in combination with chlorambucil or R-chlorambucil likewise apply for the described methods of treatment of the present invention.

The present invention further concerns the CD37 antibody as described, any of the methods as described, the kit as described, the article of manufacture as described, the pharmaceutical composition as described, and the methods of treatment as described, whereby the CD37 antibody is a chimeric antibody defined by
a) a variable heavy chain comprising the amino acid sequence shown in SEQ ID NO: 2, and
b) a variable light chain comprising the amino acid sequence shown in SEQ ID NO:4,
whereby the constant heavy and light chains are preferably of human origin.

The present invention furthermore concerns the CD37 antibody as described, any of the methods as described, the kit as described, the article of manufacture as described, the pharmaceutical composition as described, and the methods of treatment as described, whereby the antibody has a heavy chain comprising the amino acid sequence of SEQ ID NO: 5 and a light chain comprising the amino acid sequence of SEQ ID NO: 6.

The present invention furthermore concerns the CD37 antibody as described, any of the methods as described, the kit as described, the article of manufacture as described, the pharmaceutical composition as described, and the methods of treatment as described, the antibody has a heavy chain comprising the amino acid sequence of SEQ ID NO: 7 fused to SEQ ID NO: 2 and a light chain comprising the amino acid sequence of SEQ ID NO: 8 fused to SEQ ID NO: 4.

The present invention furthermore concerns the CD37 antibody as described, any of the methods as described, the kit as described, the article of manufacture as described, the pharmaceutical composition as described, and the methods of treatment as described, s whereby the antibody has a heavy chain comprising the amino acid sequence of SEQ ID NO: 9 and a light chain comprising the amino acid sequence of SEQ ID NO: 10.

The present invention furthermore concerns the CD37 antibody as described, any of the methods as described, the kit as described, the article of manufacture as described, the pharmaceutical composition as described, and the methods of treatment as described, whereby said antibody is a humanized antibody defined by frameworks supporting said CDRs that are derived from a human antibody, and wherein the constant heavy and light chains are from a human antibody.

The present invention furthermore concerns the CD37 antibody as described, any of the methods as described, the kit as described, the article of manufacture as described, the pharmaceutical composition as described, and the methods of treatment as described, whereby the antibody has a heavy chain comprising the amino acid sequence of SEQ ID NO: 11 and a light chain comprising the amino acid sequence of SEQ ID NO: 12.

The present invention furthermore concerns the CD37 antibody as described, any of the methods as described, the kit as described, the article of manufacture as described, the pharmaceutical composition as described, and the methods of treatment as described, whereby the antibody has a heavy chain comprising the amino acid sequence of SEQ ID NO: 13 and a light chain comprising the amino acid sequence of SEQ ID NO: 14.

The present invention furthermore concerns the CD37 antibody as described, any of the methods as described, the kit as described, the article of manufacture as described, the pharmaceutical composition as described, and the methods of treatment as described, whereby the CD37-positive malignancy is selected from the group consisting of: B-cell lymphomas, aggressive B-cell lymphoma, Hodgkin's disease, B-cell non-Hodgkin's lymphoma (NHL), lymphomas, Waldenstrom's macroglobulinaemia (also called lymphoplasmacytic lymphoma or immunocytoma), central nervous system lymphomas, leukemias, acute lymphoblastic leukemia (ALL), chronic lymphocytic leukemia (CLL; also termed B-cell chronic lymphocytic leukemia BCLL), hairy cell leukemia, chronic myoblastic leukemia, myelomas, multiple myeloma, T-cell lymphoma, small lymphocytic lymphoma, B-cell prolymphocytic leukemia, lymphoplasmacytic lymphoma, splenic marginal zone lymphoma, plasma cell myeloma, solitary plasmacytoma of bone, extraosseous plasmacytoma, extra-nodal marginal zone B-cell lymphoma of mucosa-associated (MALT) lymphoid tissue, nodal marginal zone B-cell lymphoma, follicular lymphoma, mantle cell lymphoma, diffuse large B-cell lymphoma, mediastinal (thymic) large B-cell lymphoma, intravascular large B-cell lymphoma, primary effusion lymphoma, Burkitt's lymphoma/leukemia, grey zone lymphoma, B-cell proliferations of uncertain malignant potential, lymphomatoid granulomatosis, and post-transplant lymphoproliferative disorder, whereby the CD37-positive malignancy is preferably a B-cell malignancy, preferably B-cell non-Hodgkin's lymphoma, B-cell chronic lymphocytic is leukemia, whereby the B-cell malignancy is most preferably chronic lymphocytic leukemia (CLL).

The practice of the present invention will employ, unless otherwise indicated, conventional techniques of medicine, pharmacy, chemistry, biology, oncology, cell biology, molecular biology, cell culture, immunology and the like which are in the skill of one in the art. These techniques are fully disclosed in the current literature.

The following examples are not limiting. They merely show possible embodiments of the invention. A person skilled in the art could easily adjust the conditions to apply it to other embodiments.

### EXAMPLE 1

### Efficacy of Antibody A2, a Chimeric Monoclonal Antibody to CD37, in Combination with Chlorambucil in a Subcutaneous Xenograft Model of the Human Follicular Lymphoma DOHH2 in C.B-17 Scid Mice

Objectives of the Study

The goal of the present study was to assess the efficacy of antibody A2 in combination with chlorambucil chemotherapy in a model of human follicular lymphoma (DOHH2) in C.B-17 scid mice.

Design of the Study

| | Number | | Dose | Schedule [days of | |
|---|---|---|---|---|---|
| Group | of mice | Compound | [mg/kg] | administration] | Route |
| 1 | 7 | NaCl (0.9%) | — | d1, d5, d8, d12, d15 | i.p. |
| 2 | 7 | antibody A2 | 10 | d1, d5, d8, d12, d15 | i.p. |
| 3 | 7 | chlorambucil | 6 | d2, d6 | i.p. |
| 4 | 7 | antibody A2 + | 10 + 6 | d1, d5, d8, d12, d15 | i.p. |
| | | chlorambucil | | d2, d6 | |

Materials and Methods

A single batch of antibody A2 was used for this study. This antibody is specific for human CD37 and does not bind to mouse CD37. Chlorambucil was purchased from Sigma. Female C.B-Igh-l^{b}/IcrTac-Prkdc^{scid} mice were used. Antibody A2 and chlorambucil were administered intraperitoneally. Tumors were established from cultured DOHH2 cells by subcutaneous injection. Tumor volumes were determined three times a week using a caliper. Body weight of the mice was measured as an indicator of tolerability of the compounds on the same days. Day 1 was the first, day 16 the last day of the study.

Main Results

Antibody A2 as a single agent significantly inhibited growth of DOHH2 follicular lymphoma and was well tolerated. Chlorambucil administered as a single agent during the first week of the study showed moderate but significant inhibition of tumor growth, but resulted in transient weight loss with recovery during the second week. Combination of antibody A2 and chlorambucil in the first week of the study, followed by single-agent antibody A2 during the second week, induced tumor regression in all animals; statistical analysis indicated a synergistic enhancement of efficacy. Combination treatment resulted in progressive body weight loss during the first week of the study, and the animals did not fully recover until the end of the experiment.

The following tables summarize the results obtained for tumor volume (median) and body weight (median) after two weeks antibody A2 therapy and/or 1 week of chlorambucil therapy (day 16 of the study).

| | | | Weight | |
|---|---|---|---|---|
| | TGI | | change | |
| Compound | [%] | p value | [%] | p value |
| Vehicle control | — | — | +4.6 | — |
| 10 mg/kg antibody A2 | 73 | 0.0009 | +3.1 | 0.8048 |
| 6 mg/kg chlorambucil | 71 | 0.0009 | −5.4 | 0.0060 |
| 10 mg/kg antibody A2 + | 105 | 0.0009 | −3.2 | 0.0021 |
| 6 mg/kg chlorambucil | | | | |

| | 10 mg/kg | p value vs | Combi- | p value vs | 6 mg/kg |
|---|---|---|---|---|---|
| | antibody | combination | nation | combination | chloram- |
| | A2 | therapy | therapy | therapy | bucil |
| TGI [%] | 73 | 0.0014 | 105 | 0.0014 | 71 |
| PR [x/7] | 0 | — | 1 | — | 0 |
| CR [x/7] | 0 | — | 6 | — | 0 |
| Weight | +3.1 | 0.0213 | −3.2 | 0.1248 | −5.4 |
| change | | | | | |
| [%] | | | | | |

Conclusions

In the DOHH2 model of follicular lymphoma, treatment with a combination of antibody A2 and chlorambucil showed high efficacy, achieving tumor regression in all animals. Compared with single-agent treatment, the drug combination resulted in synergistic enhancement of efficacy.

1. Introduction

Antibody A2 is a mouse-human chimeric Fc-engineered IgG1 antibody with high affinity for human CD37 and potent in vitro cytotoxicity (apoptosis induction, ADCC, tumor cell depletion in whole blood assays). The antibody does not cross-react with mouse CD37. The goal of the present study was to assess the efficacy of antibody A2 in combination with chlorambucil chemotherapy in a model of human follicular lymphoma (DOHH2) in C.B-17 scid mice.

1.1 Study Design

Model: Subcutaneous xenografts of the human follicular lymphoma (DOHH2) in C.B-17 scid mice

| | Number | | Dose | Schedule [days of | |
|---|---|---|---|---|---|
| Group | of mice | Compound | [mg/kg] | administration] | Route |
| 1 | 7 | NaCl (0.9%) | — | d1, d5, d8, d12, d15 | i.p. |
| 2 | 7 | antibody A2 | 10 | d1, d5, d8, d12, d15 | i.p. |
| 3 | 7 | chlorambucil | 6 | d2, d6 | i.p. |
| 4 | 7 | antibody A2 + | 10 + 6 | d1, d5, d8, d12, d15 | i.p. |
| | | chlorambucil | | d2, d6 | |

1.2 Test Compounds

Antibody A2 (10 mg/ml) was used for this experiment and formulated in a vehicle containing 25 mM citrate, 125 mM NaCl, 0.02% PS20 pH 6.2 and diluted with PBS. Chlorambucil was purchased from Sigma, dissolved in dimethylacetamide mixed with labrafill at a ratio of 1:9.

1.3 Mice

Mice were 6 week-old female C.B-Igh-l^{b}/IcrTac-Prkdc^{scid} purchased from Taconic, Denmark. After arrival, mice were allowed to adjust to ambient conditions for at least 5 days before they were used for the experiments. They were housed in Makrolon® type III cages in groups of 7 under standardized conditions at 21.5 ±1.5° C. temperatures and 55±10% humidity. Standardized diet (PROVIMI KLIBA) and autoclaved tap water were provided ad libitum. Subcutaneously implanted (under isoflurane anesthesia) microchips were used to identify each mouse. Cage cards showing the study number, the animal identification number, the compound and dose level, the administration route as well as the schedule remained with the animals throughout the study.

1.4 Establishments of Tumors, Randomization

To establish subcutaneous tumors, DOHH2 cells were harvested by centrifugation, washed and resuspended in PBS-5% FCS at 1×10⁸ cells/ml. 100 μl cell suspension containing 1×10⁷ cells was then injected subcutaneously into the right flank of the mice (1 site per mouse). Mice were randomly distributed between the treatment and the vehicle control group (10 days after cell injection) when tumors were well established and had reached volumes of 34 to 100 mm³.

1.5 Administration of Test Compound

Antibody A2 was diluted with PBS and injected intraperitoneally with a volume of 10 ml/kg.

Chlorambucil was diluted with dimethylacetamide mixed with labrafill and injected intraperitoneally with a volume of 10 ml/kg. Solutions were kept at 6° C. for a maximum of 5 days.

1.6 Monitoring Tumor Growth and Side Effects

Tumor diameters were measured three times a week (Monday, Wednesday and Friday) with a caliper. The volume of each tumor [in mm³] was calculated according to the formula “tumor volume=length*diameter²*π/6.” To monitor side effects of treatment, mice were inspected daily for abnormalities and body weight was determined three times a week (Monday, Wednesday and Friday). Animals were sacrificed when the control tumors reached a size of approximately 1000 mm³ on average. In addition, animals with tumor sizes exceeding 1.5 cm in diameter or 20% body weight loss were euthanized for ethical reasons.

TGI values were calculated as follows:

TGI=100×{1-[(treated_{final day}−treated _{day1})/(control_{final day}−control_{day1})]}

1.7 Statistical Analysis

1.7.1 Anti-Tumor Efficacy and Change of Body Weight

For the evaluation of the statistical significance of tumor inhibition a one-tailed non-parametric Mann-Whitney-Wilcoxon U-test was performed, based on the hypothesis that an effect would only be measurable in one direction (i.e. expectation of tumor inhibition but not tumor stimulation). In general, the U-test compares the ranking of the individual tumors of two groups, according to (in this study) absolute volume on a particular day (pairwise comparisons between groups). Analysis was performed on the last days of the experiment. Tumors to which the LOCF methodology was applied until the day of the statistical analysis were included in the comparison. The p-values obtained from the U-test were adjusted using the Bonferroni-Holm correction. By convention, p-values ≦0.05 indicate significance of differences. Statistical calculations were performed using GraphPad Prism Bioanalytic Software (version 5.04 for Windows, GraphPad Software, San Diego Calif. USA, www.graphpad.com).

1.7.2 Synergism of Efficacy

The statistical evaluation was performed for the parameter tumor volume at different days. All measurements were available for all treatment groups up to Days 16, i.e. there are no missing values up to Day 16.

The tumor volume was analyzed based on descriptive statistics and by using a Mixed Model for Repeated Measurements (MMRM) up to 16 days.

The number of valid observations, mean, standard deviation, median, and the geometric mean were given. Data were log-transformed to stabilize the variance over the time course. Concerning the data on the log-scale (natural logarithm, i e logarithm to the base e) mean, standard deviation, median, minimum and maximum were displayed.

All statistical analyses were exploratory, no adjustment of the significance level for multiple testing was made, i.e. all p-values reported will have to be interpreted as part of the descriptive and exploratory analyses.

After data screening, it was noticed that linearity described sufficiently well the is logarithmized tumor volume dynamic within an animal up to day 16. The repeated tumor volume measurements were analyzed after log-transformation by a linear mixed effects model for repeated measurements. For tumor volumes measured as 0 mm³ the undefined logarithm was set to 0, corresponding to a tumor volume of 1 mm³ on the original scale. Treatment, time, and interaction term treatment*time were included as fixed effects and animal was considered as a random effect. The log-transformed tumor volume at baseline was included as a covariate in the model

Yᵢⱼₖ=μ+aᵢ+dᵢⱼ+τₖ+(aτ)ᵢₖβxᵢⱼ+υᵢⱼₖ.

Where Yᵢⱼₖ is the log-transformed tumor volume at time k on animal j in treatment group i, μis the overall mean, αᵢ is a fixed effect of treatment i, dᵢⱼ is a random effect of animal j in treatment group i, τₖ is fixed effect of time k, (ατ)ₖ is a fixed interaction effect of treatment i with time k, xᵢⱼ is the log-transformed tumor volume at baseline as a covariable, and εᵢⱼₖ is random error at time k on animal j in treatment i.

Regarding the within-subject covariance matrix R, a variance components (VC) covariance matrix R(i,j)=σₖ²χ(i=j) was chosen. The VC structure was also indicated as best among of some reasonable covariance structures on the basis of the AIC criterion. An unstructured covariance (UN) matrix has not led to a positive definite Hessian matrix and could not be considered.

The covariance parameters were estimated using residual (restricted) maximum likelihood (REML). The Kenward Roger (KR) method was chosen as the denominator degrees of freedom option in SAS PROC MIXED procedure. KR works reasonably well also with more complicated covariance structures, when sample sizes are moderate to small and the design is reasonably balanced.

To assess antagonism or synergism, additive treatment effects were calculated as summation of the monotherapy effects on log-scale (log μ_{Ref}−log μ_{T}_{₁}+log μ_{Ref}−log μ_{T}_{₂}) and were compared with the effect of the corresponding combination therapy (log μ_{Ref}−log μₜ_{₁}_{T}_{₂}).

The statistical evaluation was prepared using the software package SAS version 9.2 (SAS Institute Inc., Cary N.C., USA).

2. Results

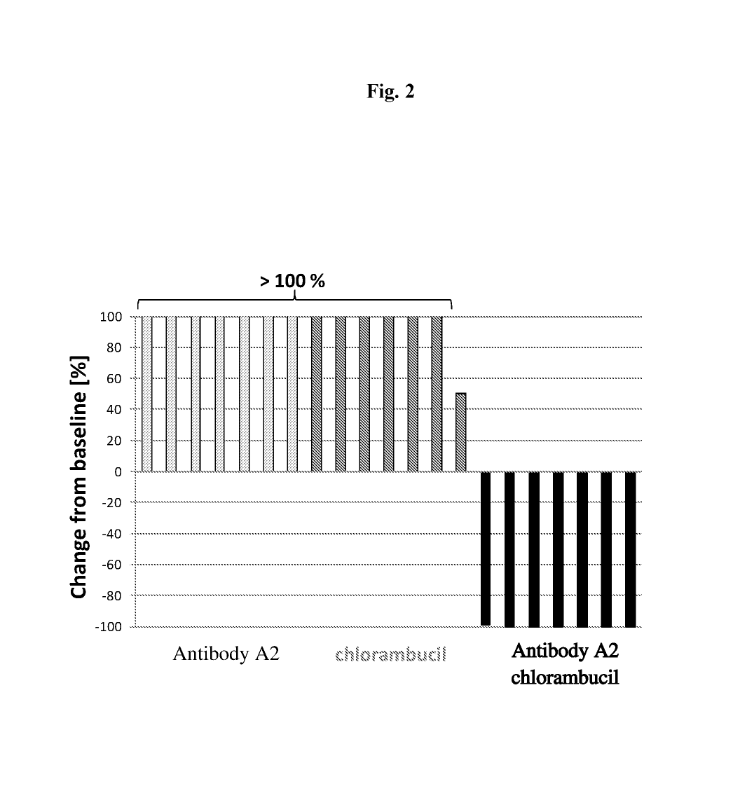

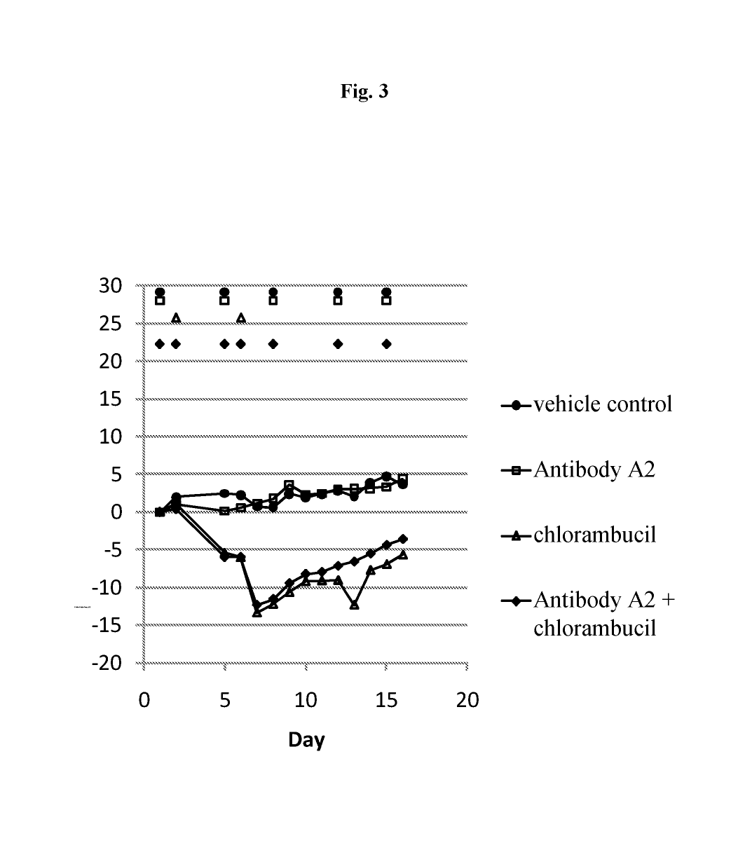

2.1 Tumor Value and Body Weight: Treatment vs Control
During the 16 day study period, control tumors grew from a median volume of 70 mm³ to a volume of 1330 mm³ at day 16 (FIG. 1, Table 1). The control animals gained 4.6% body weight (FIG. 3, Table 1).
Treatment with 10 mg/kg antibody A2 twice weekly intraperitoneally for two weeks significantly delayed tumor growth compared to the controls (median TGI=73%, p=0.0009) (FIG. 1, Table 1). Tumor regressions were not observed (FIG. 2, Table 2). Similar gain of body weight was observed compared to vehicle-treated control animals (+3.1%, p=0.8048, not significant) (FIG. 3, Table 1).
Treatment with chlorambucil administered twice (day 2 and 6) i.p. resulted in body weight loss of 13.4% after one week. Chlorambucil was therefore not administered in the second week, and the animals gained body weight, but the weight gain at the end of the experiment was still significantly different compared to the vehicle-treated control animals (−5.4%, p=0.0060) (FIG. 3, Table 1). Treatment significantly delayed tumor growth compared to the controls (median TGI=71%, p=0.0009) (FIG. 1, Table 1), but tumor regressions were not observed (FIG. 2, Table 2).
Treatment with the combination of antibody A2 (twice weekly) and chlorambucil (day 2 and 6, no administration during the second week as described above) significantly delayed tumor growth compared to the controls (median TGI=105%, p=0.0009) (FIG. 1, Table 1). 6 out of 7 tumors completely regressed and one tumor partially regressed to a volume of only 1.1 mm³ (FIG. 2, Table 2). During the first 7 days of the study, the animals progressively lost body weight (median up to 12.4%), and recovery was slow during the remainder of the experiment. At the end of the study, the loss of body weight was significantly different from the weight gain of the vehicle-treated control animals (−3.2%, p=0.0021) (FIG. 3, Table 1).

2.2 Tumor Volume and Body Weight: Combination Therapy vs Single-Agent Therapy

Two weeks of therapy with a combination of antibody A2 and chlorambucil was significantly more efficacious than single agent treatment with antibody A2 (median TGI=105% versus 73%, p=0.0014) or with chlorambucil (median TGI=105% versus 71%, p=0.0014) (FIG. 1, Table 2).

When the tumor volumes were analyzed based on descriptive statistics and by using a mixed model for repeated measurements (MMRM), combination therapy was found to show synergistic activity compared to the corresponding monotherapies from day 9 on (p<0.0001).

On day 16, the body weight change in the combination group (−3.2%) was significantly different compared to that in the antibody A2 group (+3.1%, p=0.0213), but not to the chlorambucil group (−5.4%, p=0.1248) (FIG. 3, Table 2).

3. Discussion

In the present study, two weeks of treatment with antibody A2 at a dose of 10 mg/kg twice weekly significantly delayed tumor growth (TGI=73%). Treatment was well tolerated, however, it must be noted that antibody A2 binds specifically to human CD37 and does not cross-react with mouse CD37.

Chlorambucil at 6 mg/kg was poorly tolerated during the first week of treatment; the compound was therefore not administered in the second week of the study and the animals slowly recovered from their weight loss. Treatment resulted in moderate but significant efficacy (TGI=71%).

Combination therapy during the first week of the study followed by single-agent antibody A2 during the second week showed very high efficacy (TGI=105%, 6 out of 7 complete and one partial tumor regressions) that according to statistical assessment can be considered as synergistic. Animals in the combination group markedly lost weight during the study; subsequent recovery was slow, and signficant weight loss was still observed on day 16, but the difference was not different from the weight loss of the animals treated with chlorambucil alone.

4. Conclusion

In the DOHH2 model of follicular lymphoma, treatment with a combination of antibody A2 and chlorambucil showed high efficacy, achieving tumor regression in all animals. Compared with single-agent treatment, the drug combination resulted in synergistic enhancement of efficacy.

5. Tables

TABLE 1
Tumor volume and body weight: treatment
vs. control (results on day 16)

| | | | Weight | |
|---|---|---|---|---|
| | TGI | | change | |
| Compound | [%] | p value | [%] | p value |
| Vehicle control | — | — | +4.6 | — |
| 10 mg/kg antibody A2 | 73 | 0.0009 | +3.1 | 0.8048 |
| 6 mg/kg chlorambucil | 71 | 0.0009 | −5.4 | 0.0060 |
| 10 mg/kg antibody A2 + | 105 | 0.0009 | −3.2 | 0.0021 |
| 6 mg/kg chlorambucil | | | | |

bold p value < 0.05

TABLE 2
Tumor volume and body weight: combination therapy
vs. single agent therapy (results on day 16)

| | 10 mg/kg | p value vs | Combi- | p value vs | 6 mg/kg |
|---|---|---|---|---|---|
| | antibody | combination | nation | combination | chloram- |
| | A2 | therapy | therapy | therapy | bucil |
| TGI [%] | 73 | 0.0014 | 105 | 0.0014 | 71 |
| PR [x/7] | 0 | — | 1 | — | 0 |
| CR [x/7] | 0 | — | 6 | — | 0 |
| Weight | +3.1 | 0.0213 | −3.2 | 0.1248 | −5.4 |
| change | | | | | |
| [%] | | | | | |

bold p value < 0.05

1. A method of using a CD37 antibody in combination with chlorambucil for treating patient suffering from a CD37-positive malignancy, wherein the CD37 antibody comprises:
a variable heavy chain comprising CDRs have the SEQ ID NOs: 15, 16 or 21, and 17, and
a variable light chain comprising CDRs having the SEQ ID NOs: 18, 19 and 20.

2. The method of claim 1, wherein the patient receives at least one dose of the CD37 antibody and at least one dose of chlorambucil during a treatment cycle, wherein a treatment cycle is a time period of about 1 to 6 weeks.

3. The method of claim 1, wherein the patient additionally receives at least one dose of a CD20 antibody.

4. The method of claim 1, wherein the CD37 antibody is administered to said patient simultaneously with the administration of chlorambucil.

5. The method of claim 1, wherein the CD37 antibody is administered to said patient after the administration of chlorambucil.

6. The method of claim 1, wherein the CD37 antibody is administered to said patient before the administration of chlorambucil.

7. The method of claim 1, wherein the CD37 antibody is additionally administered at least one more time during a treatment cycle.

8. The method of claim 1, wherein the said CD37 antibody is administered in a dose of about 10 μk/kg to 40 mg/kg or in a dose of about 1 mg to 2800 mg per patient.

9. The method of claim 1, wherein the estimated weekly dose of CD37 antibody for a 70 kg human is in the range of 1 mg to 2800 mg, preferably or in the range of 1 mg to 400 mg weekly, or in the range 2 mg to 800 mg every 2 weeks, wherein the CD37 antibody preferably comprises SEQ ID NOs: 5 and 6.

10. The method of claim 1, wherein the estimated weekly dose of CD37 antibody for a 70 kg human is in the range of 1 mg to 2800 mg, or in the range of 1 mg to 1000 mg, or in the range of 100 mg to 385 mg weekly, or in the range of 200 mg to 770 mg every two weeks, wherein the CD37 antibody preferably comprises SEQ ID NOs: 11 and 12.

11. The method of claim 1, wherein the dose for chlorambucil ranges between 50-150 mg/m² body surface, or between 70-120 mg/m² body surface, or between 100-150 mg/m² body surface, or between 60-70 mg/m² body surface.

12. The method of claim 1, wherein the patient is a patient suffering from CLL and wherein chlorambucil is administered at a dosage of 100 mg/m² body surface on days 1 and 2 of the treatment cycle, which treatment cycle is 3-4 weeks long.

13. The method of claim 1, wherein the patient is a patient suffering from B-cell non-Hodgkin's lymphoma (B-NHL) and wherein chlorambucil is administered at a dosage of 120 mg/m² body surface on days 1 and 2 of the treatment cycle, which treatment cycle is 3-4 weeks.

14. The method claim 1, wherein chlorambucil is administered as a one-time administration per treatment cycle with a dose of 70-400 mg/m² body surface.

15. The method claim 1, wherein the combination of the CD37 antibody and chlorambucil is administered as first line treatment.

16. The method of claim 1, wherein the combination of the CD37 antibody and chlorambucil is administered as second or later line treatment.

17. A method of reducing CD37-positive cells comprising:
a) Exposing CD37-positive cells to a CD37 antibody and
b) Exposing CD37-positive cells to chlorambucil,
wherein said CD37 antibody of step a) comprises:
i) a variable heavy chain comprising CDRs have the SEQ ID NOs: 15, 16 or 21, and 17, and
ii) a variable light chain comprising CDRs having the SEQ ID NOs: 18, 19 and 20.

18. The method of claim 17, wherein the CD37-positive cells are additionally exposed to a CD20 antibody.

19. The method of claim 17, wherein the CD37-positive cells are exposed to the CD37 antibody and chlorambucil simultaneously, or
wherein the CD37-positive cells are exposed to the CD37 antibody within 24 hrs or within 36 hrs after they are exposed to chlorambucil, or
wherein the CD37-positive cells are exposed to the CD37 antibody within 24 hrs or within 36 hrs before they are exposed to chlorambucil.

20-34. (canceled)
